(19) United States
(12) Patent Application Publication
(10) Pub. No.: US 2026/0060746 A1
(43) Pub. Date: Mar. 5, 2026

(54) SYSTEM AND METHODS FOR CUTTING HEART VALVE LEAFLETS

(71) Applicant: BOSTON SCIENTIFIC SCIMED, INC., MAPLE GROVE, MN (US)

(72) Inventors: Eric Michael Petersen, Maple Grove, MN (US); Philip Andrew Litecky, Forest Lake, MN (US); Joseph Edward Adriaens, Elk River, MN (US); Joel T. Eggert, Plymouth, MN (US); Daniel Presteng, Corcoran, MN (US)

(73) Assignee: BOSTON SCIENTIFIC SCIMED, INC., MAPLE GROVE, MN (US)

(21) Appl. No.: 19/315,426

(22) Filed: Aug. 29, 2025

Related U.S. Application Data
(60) Provisional application No. 63/689,541, filed on Aug. 30, 2024.

(51) Int. Cl. A61B 18/14; A61B 18/00

(52) U.S. Cl. CPC A61B 18/1492; A61B 2018/0022; A61B 2018/00369; A61B 2018/00601; A61B 2018/1467

(57) ABSTRACT

A system for cutting at least one valve leaflet of a heart valve having multiple valve leaflets includes an elongate shaft, an expandable balloon coupled to the elongate shaft, an expandable framework disposed radially outward of the expandable balloon, and at least one electrode disposed on the expandable framework. The expandable balloon is configured to shift between a deflated configuration and an inflated configuration, and the expandable framework is configured to shift between a collapsed configuration and an expanded configuration. When the expandable framework is in the expanded configuration, the at least one electrode is configured to cut the at least one valve leaflet.

### CROSS-REFERENCE TO RELATED APPLICATIONS

This application claims the benefit of priority of U.S. Provisional Application No. 63/689,541 filed Aug. 30, 2024, the entire disclosure of which is hereby incorporated by reference.

### TECHNICAL FIELD

The disclosure relates generally to medical devices and more particularly to medical devices that are adapted for cutting at least one valve leaflet of a heart valve (native or implanted).

### BACKGROUND

A wide variety of intracorporeal medical devices have been developed for medical use including medical devices for repair or replacement of diseased heart valves. Transcatheter aortic valve replacement (TAVR) procedures rely on the replacement heart valve implant to push aside native valve leaflets (or replacement valve leaflets in instances where a second TAVR implant is being implanted within a first TAVR implant) in order to function properly. In some instances, diseased and/or fused valve leaflets may cause challenges for transcatheter valve replacement procedures. For example, the force required to push the diseased valve leaflets aside may exceed the capabilities of the replacement heart valve implant. In some instances, balloon valvuloplasty is performed prior to implanting the replacement heart valve implant to pre-dilate the heart valve. However, balloon valvuloplasty performed in conjunction with transcatheter valve replacement procedures may present its own complications and/or risks, and in some instances, stenosis or leaflet fusion may “survive” the balloon valvuloplasty procedure and prevent the replacement heart valve implant from fully expanding, properly seating within the valve annulus, and/or functioning properly. In some cases, the previous valve leaflets, when pinned in the open position, may block the coronary artery. There is an ongoing need to provide alternative medical devices as well as alternative methods for manufacturing and using medical devices to improve the efficiency and success of TAVR procedures.

### SUMMARY

This disclosure provides design, material, manufacturing method, and use alternatives for medical devices. An example system for cutting at least one valve leaflet of a heart valve having multiple valve leaflets includes an elongate shaft, an expandable balloon coupled to a distal end of the elongate shaft, wherein the expandable balloon is configured to shift between a deflated configuration and an inflated configuration, an expandable framework disposed radially outward of the expandable balloon, wherein the expandable framework is configured to shift between a collapsed configuration and an expanded configuration, and at least one electrode disposed on the expandable framework, wherein when the expandable framework is in the expanded configuration, the at least one electrode is configured to cut the at least one valve leaflet.

Alternatively, or additionally to the embodiment above, the expandable framework includes a proximal base and at least two struts extending distally from the proximal base, wherein at least one of the struts has the at least one electrode disposed on an outer surface thereof.

Alternatively, or additionally to any of the embodiments above, the at least two struts each have an outer engagement surface configured to engage the at least one valve leaflet, wherein the at least two struts expand to a first diameter at a first location along each strut and to a second diameter at a second location spaced apart along each strut from the first location, wherein the first diameter is larger than the second diameter.

Alternatively, or additionally to any of the embodiments above, the first location is distal of the second location.

Alternatively, or additionally to any of the embodiments above, the first location is proximal of the second location.

Alternatively, or additionally to any of the embodiments above, the expandable framework includes a distal end cap, wherein the at least two struts are fixed to the distal end cap.

Alternatively, or additionally to any of the embodiments above, the at least two struts each have a first end fixed to the proximal base and a second free end.

Alternatively, or additionally to any of the embodiments above, the expandable framework includes at least three struts, and the at least three struts are spaced apart circumferentially around the proximal base.

Alternatively, or additionally to any of the embodiments above, the expandable framework includes at least three electrodes, one disposed on the outer surface of each of the at least three struts.

Alternatively, or additionally to any of the embodiments above, at least an inner surface of each of the at least two struts includes insulation.

Alternatively, or additionally to any of the embodiments above, the expandable framework is biased in the collapsed configuration.

Alternatively, or additionally to any of the embodiments above, the expandable balloon is semi-compliant and configured to provide a constantly increasing radially outward force on the expandable framework when in the inflated configuration.

Alternatively, or additionally to any of the embodiments above, the at least one electrode extends radially outward at least 0.25 mm beyond an outer surface of the expandable framework.

Another example system for cutting a valve leaflet of a heart valve having multiple valve leaflets includes an elongate shaft with an expandable balloon coupled to a distal end of the elongate shaft, wherein the expandable balloon is configured to shift between a deflated configuration and an inflated configuration, an expandable framework disposed on an outer surface of the expandable balloon, the expandable framework including a proximal base and at least two struts extending distally from the proximal base, the at least two struts spaced apart around the proximal base, the expandable framework configured to shift between a collapsed configuration and an expanded configuration, and at least one electrode disposed on a radially outward surface of at least one of the struts, wherein when the expandable framework is in the expanded configuration, the at least one electrode is configured to cut the valve leaflet.

Alternatively, or additionally to the embodiment above, the at least two struts each have an outer engagement surface configured to engage the valve leaflet, wherein the at least two struts expand to a first diameter at a first location along the outer engagement surface and to a second diameter at a second location spaced apart along the outer engagement surface from the first location, wherein the first diameter is larger than the second diameter.

Alternatively, or additionally to any of the embodiments above, the first location is distal of the second location.

Alternatively, or additionally to any of the embodiments above, the first location is proximal of the second location.

Alternatively, or additionally to any of the embodiments above, the expandable framework includes a distal end cap, wherein the at least two struts are fixed to the distal end cap.

Alternatively, or additionally to any of the embodiments above, the at least two struts each have a proximal region fixed to the proximal base and a second free end.

An example method of cutting a leaflet of an existing heart valve includes inserting a cutting device in a compressed configuration into the existing heart valve, the cutting device including an elongate shaft with an expandable balloon coupled to a distal end thereof, the expandable balloon configured to shift between a deflated configuration and an inflated configuration, an expandable framework disposed radially outward of the expandable balloon, the expandable framework configured to shift between a collapsed configuration and an expanded configuration, wherein at least one electrode is disposed on the expandable framework. The method further includes expanding the expandable balloon to expand the expandable framework against the leaflet with the at least one electrode in contact with a target location of the leaflet, activating the at least one electrode to begin cutting the leaflet, and moving the cutting device axially to continue cutting the leaflet.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a system for cutting valve leaflets;

FIG. 2A is a perspective view of an example expandable framework;

FIG. 2B illustrates the expandable framework of FIG. 2A expanded within a native aortic valve;

FIG. 3 is a cross-sectional view of cut valve leaflets in accordance with the disclosure;

FIG. 4A is a perspective view of another example expandable framework;

FIG. 4B illustrates the expandable framework of FIG. 4A expanded within a native aortic valve;

FIG. 5A is a perspective view of a further example expandable framework;

FIG. 5B illustrates the expandable framework of FIG. 4A expanded within a native aortic valve;

FIG. 6A is a perspective view of another example expandable framework;

FIG. 6B illustrates the expandable framework of FIG. 6A expanded within a native aortic valve;

FIGS. 7A and 7B illustrate another system for cutting valve leaflets disposed within a native aortic valve, in a partially expanded and fully expanded configuration, respectively;

FIGS. 8A and 8B illustrate a further example expandable framework expanded within a native aortic valve, in a partially expanded and fully expanded configuration, respectively; and

FIGS. 9A-9D illustrate a cutting sequence of the leaflet of a previously implanted stent valve in accordance with the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

For the purpose of this disclosure, the discussion herein is directed toward treating a native or existing heart valve such as the aortic valve and will be so described in the interest of brevity. This, however, is not intended to be limiting as the skilled person will recognize that the following discussion may also apply to other heart valves, and/or previously implanted replacement heart valve implants, with no or minimal changes to the structure and/or scope of the disclosure.

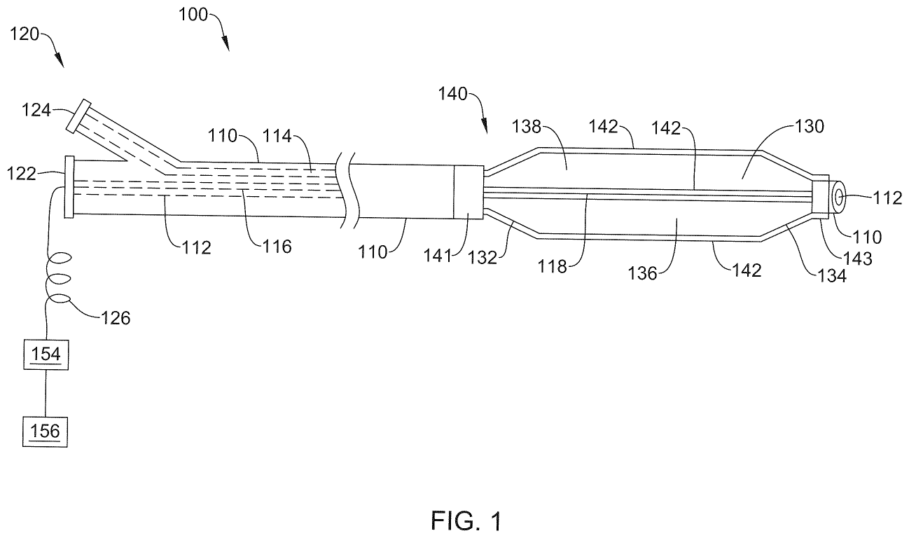

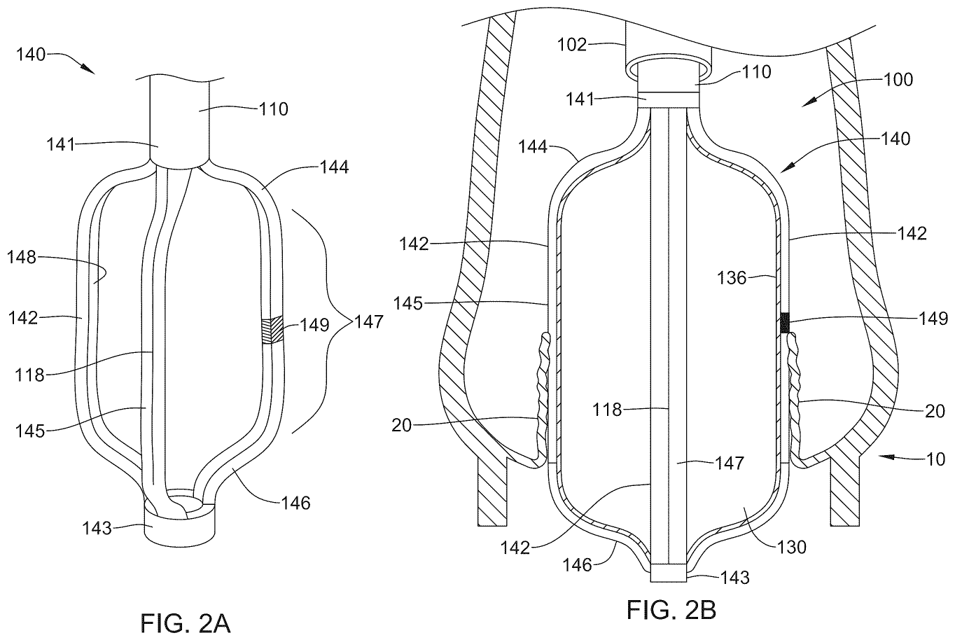

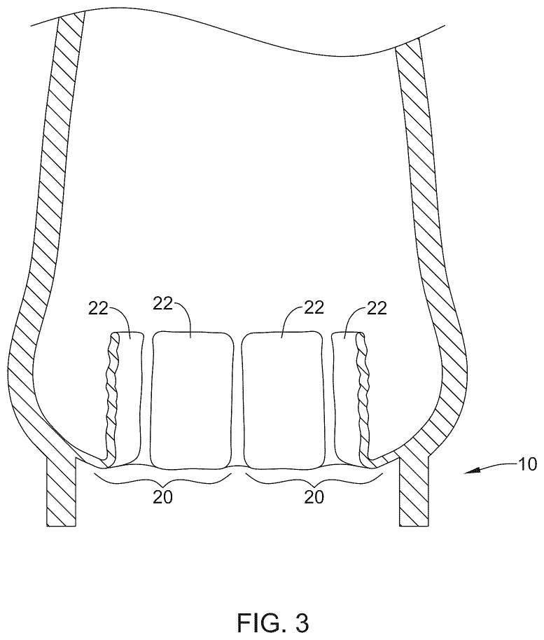

FIG. 1 illustrates selected aspects of a system 100 for cutting at least one valve leaflet 20 of a heart valve 10 (e.g., an aortic valve, a replacement heart valve implant, etc.), shown in cross-sectional view in FIGS. 2B, 3B, 4B, 5B, for example. In some embodiments, the system 100 may include an elongate shaft 110 with an expandable balloon 130 coupled to the distal end of the elongate shaft 110, and expandable framework 140 disposed radially outward of the expandable balloon 130. In some embodiments, the expandable balloon 130 may be coupled immediately adjacent to the distal end of the elongate shaft 110. In some embodiments, the expandable balloon 130 may be configured to shift between a deflated configuration (e.g., FIG. 1) and an inflated configuration (e.g., FIG. 2B) via an inflation fluid. In at least some embodiments, the expandable balloon 130 may include a proximal waist 132 fixedly attached to the elongate shaft 110, a distal waist 134 fixedly attached to the elongate shaft 110, and a body portion 136 extending between the proximal waist 132 and the distal waist 134. In some embodiments, the expandable balloon 130 may be formed from a semi-compliant or elastic material. In some embodiments, the expandable balloon 130 may be formed from an inelastic material. In at least some embodiments, the expandable balloon 130 may be formed from a polymeric material. The expandable balloon 130 will generally be at least semi-compliant. In some embodiments, the expandable balloon 130 may have an inflated diameter that is adjustable from 18-30 millimeters (mm) (0.709-1.181 inches). The expandable balloon 130 will be configured to be continually and increasingly inflated to provide a constantly increasing radially outward force on the expandable framework 140 when in the inflated configuration, thereby providing a constant force against the multiple valve leaflets. The expandable balloon 130 may be devoid of any reinforcement structures.

In some embodiments, the expandable framework 140 may be secured to the elongate shaft 110. The expandable framework 140 may be a separate, discrete piece from the elongate shaft 110 and fixedly attached to the elongate shaft 110. In some embodiments, the expandable framework 140 may be secured and/or fixedly attached to the elongate shaft 110 via mechanical fasteners, adhesive bonding, welding, mechanical interference, friction fit, etc. In other embodiments, the expandable framework 140 may be monolithically formed as a single piece with the elongate shaft 110. Other configurations are also contemplated. In at least some embodiments, the expandable framework 140 may be formed from a metallic material. In some embodiments, the expandable framework 140 may be formed from a polymeric material. In some embodiments, the expandable framework 140 may be formed from a composite material. Other configurations are also contemplated.

The expandable framework 140 may be configured to shift between a collapsed configuration (e.g., FIG. 1) and an expanded configuration (e.g., FIGS. 2A, 4A, 5A, 6A). In some embodiments, the expandable framework 140 may be biased in the collapsed configuration, and be configured to be expanded by inflating the expandable balloon 130 disposed within the expandable framework 140. In other embodiments, the expandable framework 140 may be biased in the expanded configuration. In this embodiment, the expandable framework 140 may be compressed within an outer sheath (not shown) during delivery, and upon moving distally out of the outer sheath, the expandable framework 140 self-expands to its fully expanded configuration. The expandable balloon 130 may then be inflated to provide added pressure on the expandable framework and hold it against the valve leaflets.

The expandable framework 140 may include a proximal base 141, a distal end cap 143, and at least one strut 142 extending distally from the proximal base 141 to the distal end cap 143. The proximal base 141, the distal end cap 143, and the at least one strut 142 may be formed monolithically from a single piece, such as a cut tube. Alternatively, the at least one strut 142 may be formed separately and its first end may be fixed to the proximal base 141 and a second end fixed to the distal end cap 143. The proximal base 141 and the distal end cap 143 may both extend circumferentially around and be fixed to the elongate shaft 110. In other embodiments, the proximal base 141 and the distal end cap 143 may be rotationally coupled to the elongate shaft 110. In some embodiments, the expandable framework 140 includes two struts 142, which may be disposed 180 degrees from one another. In other embodiments, the expandable framework 140 includes at least three struts 142. The struts 142 may be evenly spaced apart circumferentially around the proximal base 141. In other embodiments, four or five struts 142 may be provided. The expandable framework 140 may include at least one electrode 118 disposed thereon. The at least one electrode 118 may be positioned on a radially outward surface of the strut 142 such that when the expandable framework 140 is in the expanded configuration within a heart valve, the at least one electrode 118 may be configured to cut at least a portion of a valve leaflet. In some embodiments, the at least one electrode 118 may extend radially outward at least 0.25 mm (0.0098 inches) beyond an outer surface of the expandable framework 140.

In some embodiments at least one of the struts 142 may have an electrode 118 fixed to an outer surface of the strut 142, as shown in FIG. 1. In some embodiments, only a single strut 142 may have an electrode 118 fixed to the outer surface thereof. The electrode 118 may extend along the entire length of the strut 142, or the electrode 118 may extend over only a portion of the strut. The electrode 118 may extend in a substantially straight line along the outer surface of the strut 142. In other embodiments, multiple struts 142 may have an electrode 118 disposed thereon. In one example, when four struts 142 are present, every other strut 142 may have an electrode 118 disposed thereon, with the alternate struts being devoid of any electrodes. In still further embodiments, every strut 142 may have an electrode 118 fixed to an outer surface thereof. The electrode 118 is disposed along the outer surface of the strut(s) 142 such that when the expandable framework 140 is in the expanded configuration within the valve, the struts 142 are pressed against the leaflets, and the electrode 118 may be energized to cut the leaflet. When two or more electrodes 118 are provided on two or more struts 142, the electrodes 118 may be individually wired and may be selectively energized one at a time. In other embodiments, all of the electrodes 118 may be energized simultaneously.

The elongate shaft 110 may include a proximal manifold 120 disposed at a proximal end of the elongate shaft 110. In some embodiments, the proximal manifold 120 may include a plurality of ports. In the embodiment illustrated in FIG. 1, the proximal manifold 120 includes a guidewire port 122 in communication with a guidewire lumen 112 extending through the elongate shaft 110 and/or to a distal end of the elongate shaft 110. In some embodiments, the guidewire lumen 112 may open distally at a distalmost end of the elongate shaft 110. The proximal manifold 120 may include an inflation port 124 in fluid communication with an inflation lumen 114 extending from the inflation port 124 to the expandable balloon 130. In some embodiments, the inflation port 124 may be configured to be coupled to a source of pressurized fluid. The pressurized fluid may be inflation fluid. The inflation lumen 114 may be configured to transfer and/or transport pressurized fluid and/or inflation fluid from the source of pressurized fluid to an interior 138 of the body portion 136 of the expandable balloon 130.

The proximal manifold 120 may include an electrical connection 126. The electrical connection 126 may be configured to be operatively coupled to an energy source, such as a radiofrequency (RF) generator 154, and to a controller 156. The proximal manifold 120 and/or the elongate shaft 110 may include a conductive wire 116 extending from the electrical connection 126 to the electrode 118 on the expandable framework 140. In some embodiments, the electrode 118 may be formed by an exposed portion of the conductive wire 116 fixed on the outer surface of the strut 142. The remainder of the conductive wire 116 may be disposed within the proximal base 141 and the elongate shaft 110. In other embodiments, the electrode 118 may be a separate electrical structure fixed to the outer surface of the strut 142 and electrically coupled to the conductive wire 116. In other embodiments, the conductive wire 116 may extend along an outer surface of the proximal manifold 120 and/or the elongate shaft 110 and be covered with an insulating cover or coating. Other configurations are also contemplated. The controller 156 may operate the RF generator 154 to energize the conductive wire 116 and electrode 118 to cut the valve leaflet when the expandable framework 140 is expanded and the strut 142 with the electrode 118 is in contact with the leaflet. When multiple electrodes 118 are present on the expandable framework 140, such as on a plurality of struts 142, the controller 156 may be operated to selectively energize the electrodes 118 individually, one at a time. Alternatively, the controller may be operated to energize all of the electrodes 118 simultaneously.

The expandable framework 140 shown in FIG. 1 is illustrated in isolation in FIG. 2A in the expanded configuration, and is shown in FIG. 2B in combination with the system 100 in place within a heart valve 10. Each strut 142 may include a proximal region 144 coupled to the proximal base 141, a middle region 145, and a distal region 146 coupled to the distal end cap 143. In the expanded configuration, the proximal region 144 of each strut 142 may extend distally and radially outward from the proximal base 141 to the middle region 145. At least a portion of the middle region 145 may define an outer engagement surface 147 configured to engage the valve leaflet. An electrode 118 may be disposed on the outer engagement surface 147. In the embodiment shown in FIG. 2A, the expandable framework 140 includes three struts 142 spaced apart circumferentially around the proximal base 141, and each strut 142 has an electrode 118 disposed on the outer engagement surface 147. In this embodiment, three electrodes 118 are available for cutting the leaflets. In some embodiments the inner surface of at least those struts 142 carrying an electrode 118 may have a layer of insulation 148. Insulation may also cover a portion of the conductive wire 116 extending along the proximal region 144 and in some cases, a portion of the outer engagement surface 147, in order to provide the electrode 118 only on a discrete portion of the strut 142.

The middle region 145 may extend substantially parallel to a longitudinal axis extending centrally through the proximal base 141 and the distal end cap 143. The middle region 145 may have a uniform diameter along its length when the expandable framework 140 is in the expanded configuration. In other embodiments, the diameter may vary along the middle region 145, with a first diameter at a first location and a second diameter at a second location spaced apart along the middle region 145, where the first diameter is larger than the second diameter. The first location may be distal of the second location, which results in the cusp of the valve leaflet being cut first. Alternatively, the first location may be proximal of the second location, which results in the free edge of the valve leaflet being cut first. The distal region 146 of each strut 142 may extend distally and radially inward from the middle region 145 to the distal end cap 143. As shown in FIGS. 2A and 2B, the proximal region 144 may slope downward between the proximal base 141 and the middle region 145.

In some embodiments, the system 100 may include a delivery catheter 102, as seen in FIGS. 2B, 4B, 5B, and 6B. In some embodiments, the elongate shaft 110, the expandable balloon 130, and/or the expandable framework 140 may be axially slidable within a lumen of the delivery catheter 102. In some embodiments, the delivery catheter 102 may include an aspiration function and/or capability. In some embodiments, the system 100 may include a separate aspiration catheter delivered and/or extending alongside the elongate shaft 110. Other configurations are also contemplated. In some embodiments, the system 100 may be devoid of the delivery catheter 102.

In use, the system 100 may be advanced through the patient's vasculature to a position adjacent a heart valve 10 and/or a treatment site, as shown in FIG. 2B. In some embodiments, the expandable balloon 130 and the expandable framework 140 may be deployed from a distal end of the delivery catheter 102. In at least some embodiments, the expandable balloon 130 may be shifted from the deflated configuration (e.g., FIG. 1) toward and/or to the inflated configuration (e.g., FIGS. 2B, 4B, 5B, 6B) within the heart valve and/or the treatment site. In some embodiments, shifting the expandable balloon 130 from the deflated configuration toward and/or to the inflated configuration may shift the expandable framework 140 from the collapsed configuration toward and/or to the expanded configuration via an inflation fluid introduced through the inflation port 124.

In some embodiments, one or more visualization means and/or methods may be used to align the at least one strut 142 with the at least one valve leaflet 20 of the heart valve 10 before and/or during inflation of the expandable balloon 130 (e.g., shifting the expandable balloon 130 from the deflated configuration toward and/or to the inflated configuration). In some embodiments, the system 100 and/or the elongate shaft 110 may include a marker element (not shown) configured to align the electrode 118 on at least one strut 142 with the at least one valve leaflet 20 of the heart valve 10. In the embodiment shown in FIGS. 2A and 2B, a marker 149, such as a radiopaque band, may be positioned along the outer engagement surface 147 of the strut. During use, the heart valve 10 may be imaged using convention means and fluoroscopy may be used to align the marker 149 relative to the annulus of the heart valve 10. The marker 149 on the strut 142, or any other marker on the elongate shaft 110 or other part of the system 100, may be used to align the electrode 118 on at least one strut 142 with the at least one valve leaflet 20 of the heart valve 10 before and/or during inflation of the expandable balloon 130 (e.g., shifting the expandable balloon 130 from the deflated configuration toward and/or to the inflated configuration). In some alternative configurations, the expandable framework 140 may be shifted from the collapsed configuration toward and/or to the expanded configuration without shifting the expandable balloon 130 from the deflated configuration toward and/or to the inflated configuration. For example, the expandable framework 140 may be made of nitinol or other material providing a biased expanded configuration, such that the expandable framework 140 automatically expands to the expanded configuration upon exit from the delivery catheter 102.

In some embodiments, shifting the expandable balloon 130 from the deflated configuration toward and/or to the inflated configuration may deflect and/or urge the at least one valve leaflet 20, and/or each valve leaflet of the at least one valve leaflet 20, toward an open position wherein the at least one valve leaflet 20, and/or each valve leaflet of the at least one valve leaflet 20, extends downstream from an annulus of the heart valve 10. The at least one electrode 118, may be substantially aligned with the at least one valve leaflet 20, and/or each valve leaflet of the at least one valve leaflet 20, as shown in FIG. 2B. When the electrode 118 extends the entire length of the strut 142, the system 100 will achieve leaflet cutting regardless of how far into the heart valve 10 the expandable framework 140 extends. In embodiments in which the electrode 118 is a discrete element with a length shorter than the length of the outer engagement surface 147, the marker 149 may be positioned adjacent one end of the outer engagement surface 147, or the marker 149 may be positioned in the center of the outer engagement surface 147 to provide guidance in positioning the expandable framework 140 in the desired axial position within the heart valve 10. In embodiments in which the electrode 118 extends the entire length of the outer engagement surface 147, the marker 149 may be positioned only on the inner surface of the strut 142, or it may be a circumferential marker surrounding the strut and positioned under the electrode 118 so as to not interfere with the electrode 118 cutting the tissue.

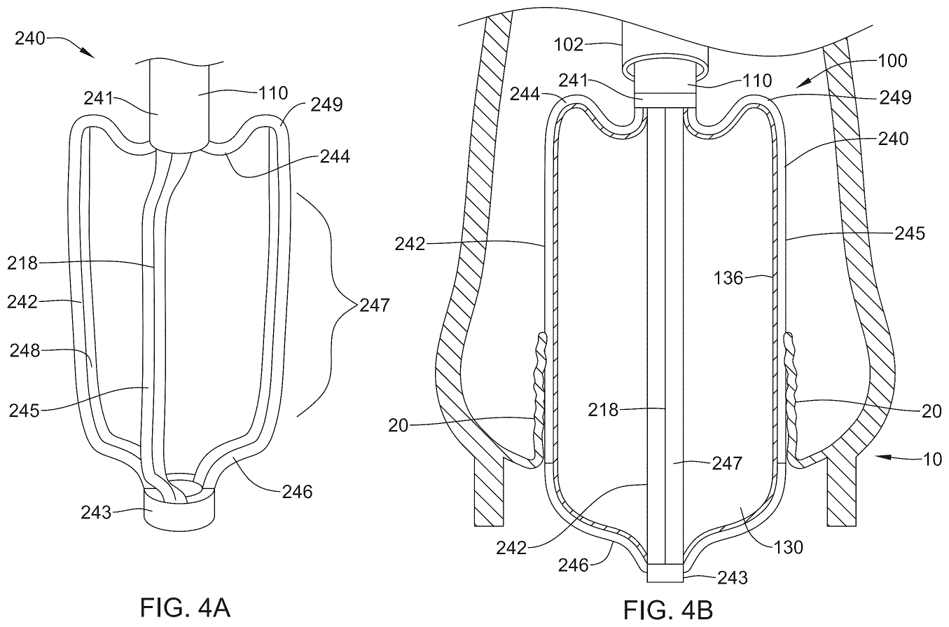

The expandable framework 140 with the electrode(s) 118 may be configured to cut (e.g., separate, resect, divide, incise, split, etc.) the at least one valve leaflet 20 (e.g., FIGS. 2B, 4B, 5B, 6B, 7, 8) of the heart valve 10 into at least two discrete pieces 22, as seen in FIG. 3 for example. In some embodiments, the at least one valve leaflet 20, and/or each leaflet of the at least one valve leaflet 20, may be cut and/or slit in a generally straight line. In some embodiments, a portion or portions of the at least one valve leaflet 20, and/or each leaflet of the at least one valve leaflet 20, may be excised or removed entirely. When a portion of a valve leaflet is to be removed entirely, a grasping device may be inserted alongside the system 100 into the region of the heart valve 10 and may be used to grasp the leaflet during excision, and then remove the excised portion. In some embodiments, the at least one electrode 118 disposed on the outer surface of the strut 142 of the expandable framework 140, as seen in FIG. 2, may be configured to cut (e.g., separate, resect, divide, incise, split, etc.) the at least one valve leaflet 20, and/or each valve leaflet of the at least one valve leaflet 20 into the plurality of discrete pieces 22, as seen in FIG. 3.

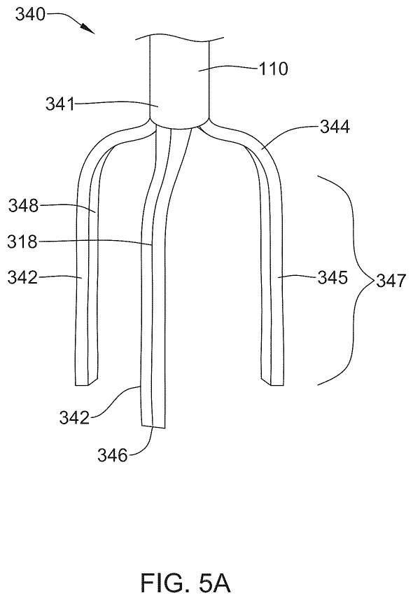

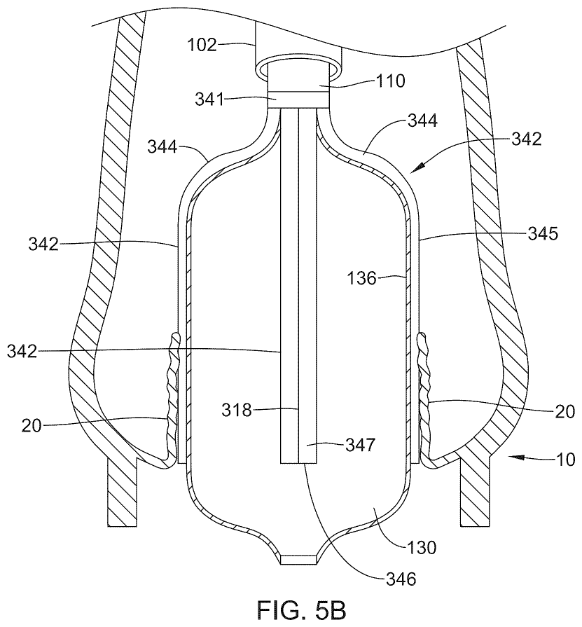

FIGS. 4A and 4B illustrate selected aspects of an alternative configuration for the expandable framework 240. The expandable framework 240 is illustrated in isolation in FIG. 4A in the expanded configuration, and is shown in FIG. 4B in combination with the system 100 in place within a heart valve 10. Similar to the expandable framework 140, the expandable framework 240 may include at least one strut 242. In some embodiments, a single strut 242 is present. In other embodiments, a plurality of struts 242 (e.g., two or more struts) are present. The expandable framework 240 as shown includes three struts 242 equally spaced apart around the circumference of the expandable framework 240. The expandable framework 240 is the same as the expandable framework 140 described above in all aspects except for the shape of the proximal region 244 of the struts 242. Each strut 242 may include a proximal region 244 coupled to the proximal base 241, a middle region 245, and a distal region 246 coupled to the distal end cap 243. In the expanded configuration, the proximal region 244 of each strut 242 may initially extend distally from the proximal base 241 and then bend back and extend proximally to a proximal tip 249 before bending and extending distally into the middle region 245. The proximal tip 249 may be at the same axial position as the distal end of the proximal base 241, or the proximal tip 249 may be disposed proximal of the distal end of the proximal base 241. In still other embodiments, the proximal tip 249 may be disposed distal of the distal end of the proximal base 241. The proximal tip 249 may extend radially outward further than the middle region 245 of the strut 242, such that the proximal tip 249 defines the radial outermost extent of the expandable framework 240 when in the expanded configuration. When the expandable framework 240 including at least two struts 242 is in the expanded configuration, the expandable framework 240 expands to a first diameter (at the proximal tip 249) at a first location along the strut 242 and to a second diameter at a second location spaced apart along the strut 242 from the first location, where the first diameter is larger than the second diameter, and the first location is proximal of the second location.

At least a portion of the middle region 245 of the strut 242 may define an outer engagement surface 247 configured to engage the valve leaflet. The middle region 245 may curve radially inward from the proximal tip 249 and then extend substantially parallel to a longitudinal axis extending centrally through the proximal base 241 and the distal end cap 243. The distal region 246 of each strut 242 may extend distally and radially inward from the middle region 245 to the distal end cap 243. As shown in FIGS. 4A and 4B, the proximal region 244 may initially slope downward from the proximal base 241 and then slope upward to the proximal tip 249 before turning back distally to the middle region 245. The proximal tip 249 may define an inverted C shape connecting a series of curves between the proximal base 241 and the middle region 245, such that no sharp corners are formed.

An electrode 218 may be disposed on the outer engagement surface 247. In the embodiment shown in FIG. 4A, the expandable framework 240 includes three struts 242 spaced apart circumferentially around the proximal base 241, and each strut 242 may have an electrode 218 disposed on the outer engagement surface 247. In some embodiments the inner surface of at least those struts 242 carrying an electrode 218 may have a layer of insulation 248. Insulation may also cover a portion of the outer engagement surface 247, in order to provide the electrode 218 only on a discrete portion of the strut 242.

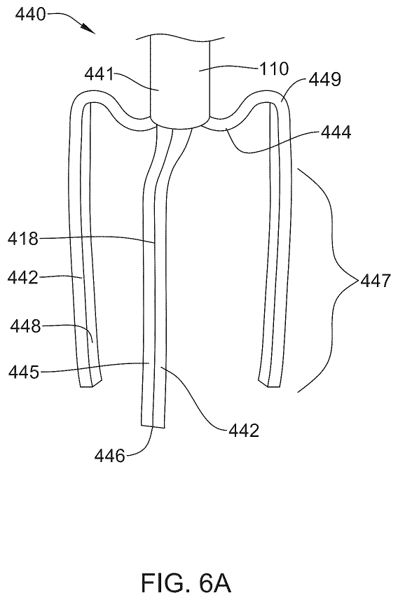

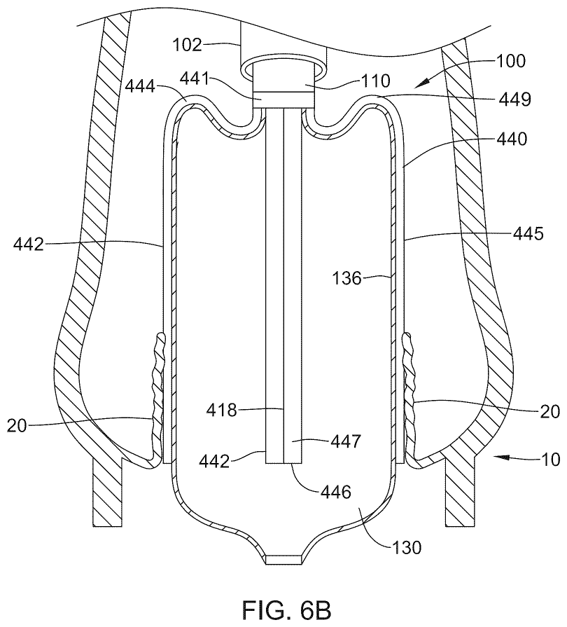

Another embodiment of expandable framework 340 is illustrated in isolation in FIG. 5A in the expanded configuration, and is shown in FIG. 5B in combination with the system 100 in place within a heart valve 10. The expandable framework 340 may have the same structure as the expandable framework 140 shown in FIGS. 2A and 2B with the exception of the distal end cap 143 and distal region 146. Expandable framework 340 lacks a distal end cap and instead, each strut 342 has a free second or distal end 346 at the distal end of the middle region 345. Each strut 342 may include a first end or proximal region 344 coupled to the proximal base 341, a middle region 345, and a free second or distal end 346. In the expanded configuration, the proximal region 344 of each strut 342 may extend distally and radially outward from the proximal base 341 to the middle region 345. At least a portion of the middle region 345 may define an outer engagement surface 347 configured to engage the valve leaflet. The middle region 345 may extend substantially parallel to a longitudinal axis extending centrally through the proximal base 341. The middle region 345 may have a uniform diameter along its length when the expandable framework 340 is in the expanded configuration. As shown, the proximal region 344 may slope downward between the proximal base 341 and the middle region 345.

An electrode 318 may be disposed on the outer engagement surface 347. In the embodiment shown in FIG. 5A, the expandable framework 340 includes three struts 342 spaced apart circumferentially around the proximal base 341, and each strut 342 may have an electrode 318 disposed on the outer engagement surface 347. In some embodiments the inner surface of at least those struts 342 carrying an electrode 318 may have a layer of insulation 348. Insulation may also cover a portion of the outer engagement surface 347, in order to provide the electrode 318 only on a discrete portion of the strut 342.

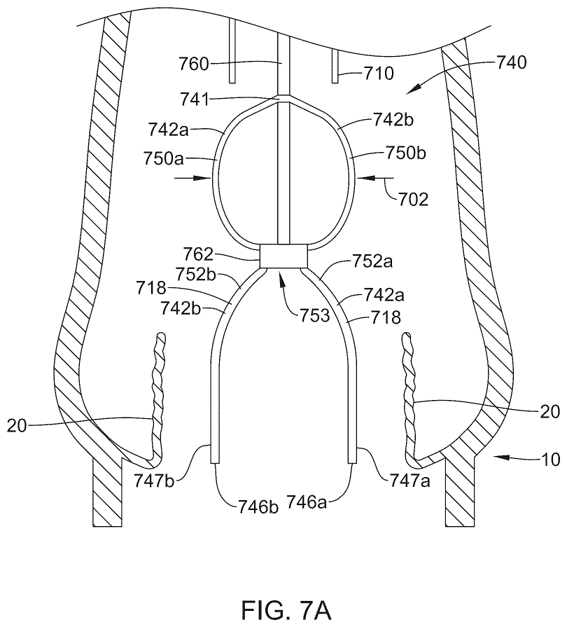

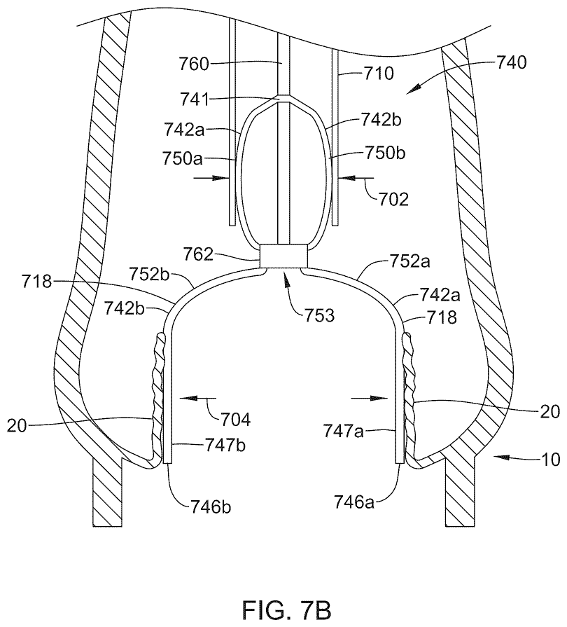

Another embodiment of expandable framework 440 is illustrated in isolation in FIG. 6A in the expanded configuration, and is shown in FIG. 6B in combination with the system 100 in place within a heart valve 10. The expandable framework 440 may have the same structure as the expandable framework 240 shown in FIGS. 4A and 4B with the exception of the distal end cap 243 and distal region 246. Expandable framework 440 lacks a distal end cap and instead, each strut 442 has a free distal end 446 at the distal end of the middle region 445.

In the expanded configuration, the proximal region 444 of each strut 442 may initially extend distally from the proximal base 441 and then bend back and extend proximally to a proximal tip 449 before bending and extending distally into the middle region 445. The proximal tip 449 may be at the same axial position as the distal end of the proximal base 441, or the proximal tip 449 may be disposed proximal of the distal end of the proximal base 441. In still other embodiments, the proximal tip 449 may be disposed distal of the distal end of the proximal base 441. The proximal tip 449 may extend radially outward further than the middle region 445 of the strut 442, such that the proximal tip 449 defines the radial outermost extent of the expandable framework 440 when in the expanded configuration. When the expandable framework 440 including at least two struts 442 is in the expanded configuration, the expandable framework 440 expands to a first diameter (at the proximal tip 449) at a first location along the strut 442 and to a second diameter at a second location spaced apart along the strut 442 from the first location, where the first diameter is larger than the second diameter, and the first location is proximal of the second location.

At least a portion of the middle region 445 of the strut 442 may define an outer engagement surface 447 configured to engage the valve leaflet. The middle region 445 may curve radially inward from the proximal tip 449 and then extend substantially parallel to a longitudinal axis extending centrally through the proximal base 441. As shown in FIGS. 6A and 6B, the proximal region 444 may initially slope downward from the proximal base 441 and then slope upward to the proximal tip 449 before turning back distally to the middle region 445. The proximal tip 449 may define an inverted C shape connecting a series of curves between the proximal base 441 and the middle region 445, such that no sharp corners are formed.

An electrode 418 may be disposed on the outer engagement surface 447. In the embodiment shown in FIG. 6A, the expandable framework 440 includes three struts 442 spaced apart circumferentially around the proximal base 441, and each strut 442 may have an electrode 418 disposed on the outer engagement surface 447. In some embodiments the inner surface of at least those struts 442 carrying an electrode 418 may have a layer of insulation 448. Insulation may also cover a portion of the outer engagement surface 447, in order to provide the electrode 418 only on a discrete portion of the strut 442.

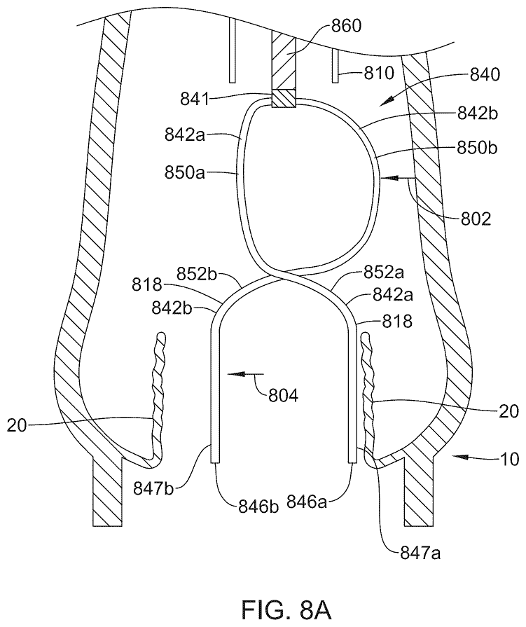

FIGS. 7A and 7B illustrate another embodiment of an expandable framework 740 coupled to an elongate shaft 760 and actuated by an actuation shaft 710 extending over the elongate shaft 760. FIG. 7A shows the expandable framework 740 in the unexpanded configuration disposed within the valve leaflets 20 of a heart valve 10. The expandable framework 740 includes a first strut 742a and a second strut 742b each fixed to a proximal base 741 which is fixed to the elongate shaft 760. Each of the first and second struts 742a, 742b includes a first curved portion 750a, 750b that extends distally and radially outward from the proximal base 741 and then curves distally and radially inward until the first and second struts 742a, 742b cross one another at crossover point 753 within a ring 762 fixed to the distal end of the elongate shaft 760. The ring 762 may be disposed circumferentially around the first and second struts 742a, 742b at the cross-over point 753. As each of the first and second struts 742a, 742b extends out the distal end of the ring 762, it forms a second curved portion 752a, 752b that extends distally and radially outward from the cross-over point 753 and then extends distally in a substantially straight or linear manner defining an outer engagement region 747a, 747b to a free end 746a, 746b. One or both of the outer engagement regions 747a, 747b of the first and second struts 742a, 742b may have an electrode 718 disposed on the radially outer surface thereof, in the same manner and with the same structure as the electrode(s) 118 on the struts 142 described above.

The expandable framework 740 may further include an actuation shaft 710 slidably disposed over the elongate shaft 760. The actuation shaft 710 is configured to slide over the first curved portions 750a, 750b and push them radially inward, as shown in FIG. 7B. Due to the somewhat S shaped curves of each of the first and second struts 742a, 742b, when the actuation shaft 710 is moved distally, it pushes the first curved portions 750a, 750b radially inward, indicated by arrows 702, which causes the second curved portions 752a, 752b to move radially outward, as indicated by arrows 704, which causes the outer engagement regions 747a, 747b to apply pressure on the valve leaflets 20, bringing the electrode(s) 718 on the outer engagement regions 747a, 747b into contact with the valve leaflet(s) 20. Conversely, when the actuation shaft 710 is moved proximally off the first curved portions 750a, 750b, the first curved portions 750a, 750b are allowed to expand radially outward which reduces the distance between the outer engagement regions 747a, 747b, and releases the expandable framework 740 from the valve leaflets 20, as shown in FIG. 7A. The curves in the first and second struts 742a, 742b are fixed such that movement of the first curved portions 750a, 750b in an inward radial direction causes the second curved portions 752a, 752b to move in an outward radial direction. The first and second struts 742a, 742b are biased with the first curved portions 750a, 750b expanded and the second curved portions 752a, 752b contracted, as shown in FIG. 7A.

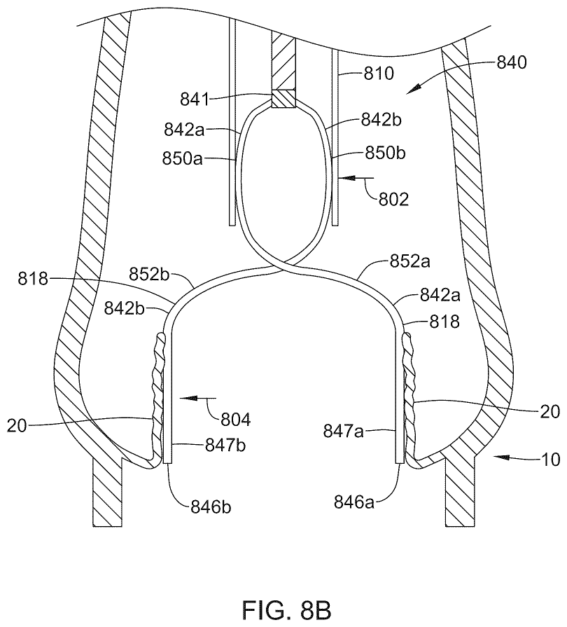

FIGS. 8A and 8B illustrate another embodiment of an expandable framework 840 coupled to an elongate shaft 860 and actuated by an actuation shaft 810 extending over the elongate shaft 860. FIG. 8A shows the expandable framework 840 in the unexpanded configuration disposed within the valve leaflets 20 of a heart valve 10. The expandable framework 840 includes a first strut 842a and a second strut 842b each fixed to a proximal base 841 which is fixed to the elongate shaft 860. The first strut 842a includes a first portion 851a that extends distally from the proximal base 841 and then extends radially inward, and the second strut 842b includes a first curved portion 850b that extends distally and radially outward from the proximal base 841 and then curves distally and radially inward until the first and second struts 842a, 842b cross one another at crossover point 853. After the crossover point 853, each of the first and second struts 842a, 842b forms a second curved portion 852a, 852b that extends distally and radially outward from the cross-over point 853 and then extends distally in a substantially straight or linear manner defining an outer engagement region 847a, 847b to a free end 846a, 846b. One or both of the outer engagement regions 847a, 847b of the first and second struts 842a, 842b may have an electrode 818 disposed on the radially outer surface thereof, in the same manner and with the same structure as the electrode(s) 118 on the struts 142 described above.

The expandable framework 840 may further include an actuation shaft 810 slidably disposed over the elongate shaft 860. When the expandable framework 840 is inserted into the valve 10, the second curved portion 852a of the first strut 842a may be positioned against one valve leaflet 20. The actuation shaft 810 is configured to slide over the first portion 851a and first curved portion 850b and push the first curved portion 850b radially inward, as shown in FIG. 8B. Due to the somewhat S shaped curve of the second strut 842b, when the actuation shaft 810 is moved distally, it pushes the first curved portion 850b radially inward, indicated by arrow 802, which causes the second curved portion 852b of the second strut 842b to move radially outward, as indicated by arrow 804, which causes the outer engagement region 847b to apply pressure on the valve leaflet 20, bringing the electrode(s) 818 on the outer engagement regions 847a, 847b into contact with the valve leaflet(s) 20. Conversely, when the actuation shaft 810 is moved proximally off the first and second struts 842a, 842b, the first curved portion 850b is allowed to expand radially outward which reduces the distance between the outer engagement regions 847a, 847b, and releases the expandable framework 840 from the valve leaflets 20, as shown in FIG. 8A. The curves in the first and second struts 842a, 842b are fixed such that movement of the first curved portion 850b in an inward radial direction causes the second curved portion 852b to move in an outward radial direction. The first and second struts 842a, 842b are biased with the first curved portion 850b expanded and the second curved portions 852a, 852b contracted, as shown in FIG. 8A.

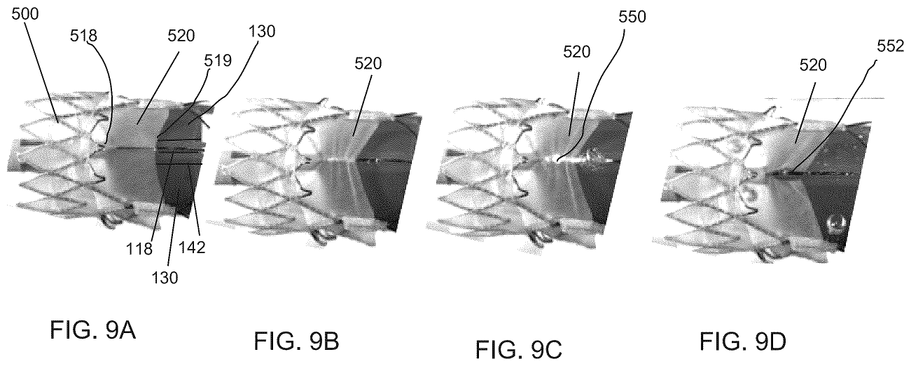

While FIGS. 2B, 3, 4B, 5B, 6B, 7, and 8 illustrate valve leaflets 20 in the native heart valve 10 as the target for cutting by the system 100 described herein, it will be understood that the system 100 may also be used to cut one or more leaflets 520 of a previously implanted artificial valve, such as the stent-valve 500 shown in FIGS. 9A-9D. All embodiments of the system 100 described herein will be understood to function equally well disposed within and cutting the native heart valve leaflets 20 and the valve leaflets 520 of any previously implanted replacement heart valve, whether synthetic or animal tissue.

FIGS. 9A-9D illustrate a method of cutting an artificial valve leaflet 520 using the system 100 with any of the expandable frameworks 140, 240, 340, 440, 740, 840 described above. The method steps apply equally to cutting the leaflet of a native heart valve. For the sake of brevity, the method will be described with the strut 142 from the expandable framework 140 shown in FIGS. 1-2B. In FIG. 9A, the system 100, which acts as a cutting device, has been inserted into the previously implanted stent-vale 500 in a compressed configuration. The expandable balloon 130 with the expandable framework 140 is positioned between the valve leaflets 520 with the electrode 118 in position at a target location of the leaflet. The expandable balloon 130 is then expanded to expand the expandable framework against the leaflet, thereby bring the struts 142 with their electrode(s) into contact with the target location on the valve leaflets 520. In the illustrated embodiment, the electrode 118 is positioned adjacent the cusp 518, distal of the leaflet free edge 519. In FIG. 9B, the electrode 118 has been activated and heating and desiccation of the valve leaflet 520 has begun. In FIG. 9C, cutting of the valve leaflet 520 has begun, creating a puncture 550 through the leaflet 520. Once cutting has begun, the expandable framework 140 is moved axially to continue cutting the leaflet. In the illustrated example, the expandable framework 140 is moved proximally by pulling the elongate shaft proximally. This moves the electrode from the cusp 518 to the leaflet free edge 519 to complete the cut 552 of the valve leaflet 520, as shown in FIG. 9D. In other embodiments, the expandable framework 140 may be initially positioned with the electrode 118 adjacent the leaflet free edge 519, and once the electrode is activated, the expandable framework 140 may be moved distally in order to cut the leaflet from the free edge 519 to the cusp 518. Regardless of which end of the leaflet is cut first, but particularly when the free edge is cut first, the expandable balloon 130 needs to keep expanding to provide a consistent force against the leaflet in order to keep the electrode 118 in contact to continue cutting. An advantage of cutting the leaflet from the free edge to the cusp is that upon initial activation of the electrode 118, a cut is begun in the leaflet, which reduces strain on the leaflet. As such, even if the cut is not completed all the way to the cusp, any degree of cut in the leaflet free edge will provide some expansion or dilation of the existing valve leaflets. Additionally, further expansion of the expandable balloon 130 may cause a partial cut to tear down to the cusp, thereby providing a complete cut even in the event of the electrode 118 not making a complete cut.

The materials that can be used for the various components of the system 100 (and/or other systems or components disclosed herein) and the various elements thereof disclosed herein may include those commonly associated with medical devices. For simplicity purposes, the following discussion refers to the system 100 (and variations, systems or components disclosed herein). However, this is not intended to limit the devices, components, and methods described herein, as the discussion may be applied to other elements, members, components, or devices disclosed herein, such as, but not limited to, the elongate shaft, the expandable balloon, the expandable framework, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, 316LV, 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antincoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A system for cutting at least one valve leaflet of a heart valve having multiple valve leaflets, the system comprising:
an elongate shaft;
an expandable balloon coupled to a distal end of the elongate shaft, wherein the expandable balloon is configured to shift between a deflated configuration and an inflated configuration;
an expandable framework disposed radially outward of the expandable balloon, wherein the expandable framework is configured to shift between a collapsed configuration and an expanded configuration; and
at least one electrode disposed on the expandable framework, wherein when the expandable framework is in the expanded configuration, the at least one electrode is configured to cut the at least one valve leaflet.

2. The system of claim 1, wherein the expandable framework includes a proximal base and at least two struts extending distally from the proximal base, wherein at least one of the struts has the at least one electrode disposed on an outer surface thereof.

3. The system of claim 2, wherein the at least two struts each have an outer engagement surface configured to engage the at least one valve leaflet, wherein the at least two struts expand to a first diameter at a first location along each strut and to a second diameter at a second location spaced apart along each strut from the first location, wherein the first diameter is larger than the second diameter.

4. The system of claim 3, wherein the first location is distal of the second location.

5. The system of claim 3, wherein the first location is proximal of the second location.

6. The system of claim 2, wherein the expandable framework includes a distal end cap, wherein the at least two struts are fixed to the distal end cap.

7. The system of claim 2, wherein the at least two struts each have a first end fixed to the proximal base and a second free end.

8. The system of claim 2, wherein the expandable framework includes at least three struts, and the at least three struts are spaced apart circumferentially around the proximal base.

9. The system of claim 8, wherein the expandable framework includes at least three electrodes, one disposed on the outer surface of each of the at least three struts.

10. The system of claim 2, wherein at least an inner surface of each of the at least two struts includes insulation.

11. The system of claim 1, wherein the expandable framework is biased in the collapsed configuration.

12. The system of claim 1, wherein the expandable balloon is semi-compliant and configured to provide a constantly increasing radially outward force on the expandable framework when in the inflated configuration.

13. The system of claim 1, wherein the at least one electrode extends radially outward at least 0.25 mm beyond an outer surface of the expandable framework.

14. A system for cutting a valve leaflet of a heart valve having multiple valve leaflets, the system comprising:
an elongate shaft with an expandable balloon coupled to a distal end of the elongate shaft, wherein the expandable balloon is configured to shift between a deflated configuration and an inflated configuration;
an expandable framework disposed on an outer surface of the expandable balloon, the expandable framework including a proximal base and at least two struts extending distally from the proximal base, the at least two struts spaced apart around the proximal base, the expandable framework configured to shift between a collapsed configuration and an expanded configuration; and
at least one electrode disposed on a radially outward surface of at least one of the struts, wherein when the expandable framework is in the expanded configuration, the at least one electrode is configured to cut the valve leaflet.

15. The system of claim 14, wherein the at least two struts each have an outer engagement surface configured to engage the valve leaflet, wherein the at least two struts expand to a first diameter at a first location along the outer engagement surface and to a second diameter at a second location spaced apart along the outer engagement surface from the first location, wherein the first diameter is larger than the second diameter.

16. The system of claim 15, wherein the first location is distal of the second location.

17. The system of claim 15, wherein the first location is proximal of the second location.

18. The system of claim 14, wherein the expandable framework includes a distal end cap, wherein the at least two struts are fixed to the distal end cap.

19. The system of claim 14, wherein the at least two struts each have a proximal region fixed to the proximal base and a second free end.

20. A method of cutting a leaflet of an existing heart valve, comprising:
inserting a cutting device in a compressed configuration into the existing heart valve, the cutting device including:
an elongate shaft with an expandable balloon coupled to a distal end thereof, the expandable balloon configured to shift between a deflated configuration and an inflated configuration;
an expandable framework disposed radially outward of the expandable balloon, the expandable framework configured to shift between a collapsed configuration and an expanded configuration, wherein at least one electrode is disposed on the expandable framework;
expanding the expandable balloon to expand the expandable framework against the leaflet with the at least one electrode in contact with a target location of the leaflet;
activating the at least one electrode to begin cutting the leaflet; and
moving the cutting device axially to continue cutting the leaflet.
